(19) United States
(12) Patent Application Publication
(10) Pub. No.: US 2020/0261013 A1
(43) Pub. Date: Aug. 20, 2020

(54) COGNITIVE AND PHYSIOLOGICAL MONITORING AND ANALYSIS FOR CORRELATION FOR MANAGEMENT OF COGNITIVE IMPAIRMENT RELATED CONDITIONS

(71) Applicant: Ilan BEN-OREN, Modi'in (IL)

(72) Inventor: Ilan BEN-OREN, Modi'in (IL)

(21) Appl. No.: 16/648,381

(22) PCT Filed: Sep. 20, 2018
(86) PCT No.: PCT/IL2018/051065

§ 371 Date: Mar. 18, 2020

Related U.S. Application Data
(60) Provisional application No. 62/563,730, filed on Sep. 27, 2017.

(51) Int. Cl. A61B 5/00; A61B 5/11; G16H 50/30; G16H 20/30; G16H 20/60; G16H 20/70

(52) U.S. Cl. CPC A61B 5/4088; A61B 5/4842; A61B 5/7275; A61B 5/7282; G16H 20/70; A61B 5/11; G16H 50/30; G16H 20/30; G16H 20/60; A61B 5/746

(57) ABSTRACT

There is provided herein systems and methods for managing a subject suffering from cognitive impairment, the method comprising: providing to a subject a cognitive training session; determining at least one aspect of the subject's cognitive performance based on and/or in response to said training session; monitoring one or more life style, physiological and/or medical parameters of the subject before, during and/or after said training session; identifying peaks in the subject's cognitive performance; wherein the identifying comprises comparing the determined cognitive performance to stored cognitive performance data; identifying changes in one or more life style, physiological and/or medical parameters positively or negatively associated with the peak in the cognitive performance; and providing the subject with a life style, physiological and/or medical recommendation based on the on identified association.

### FIELD OF THE INVENTION

Embodiments of the disclosure relate to cognitive impairment in elderly patients and/or in patients with related diseases and conditions that are associated with acute cognitive deterioration and/or fluctuations in cognitive performances.

### BACKGROUND

Cognitive impairment collectively refers to disorders manifested by a cognitive decline and typically involves decreased immediate and short-term memory, intelligence, attention, concentration, alertness, hand to eye coordination, complex problem solving ability, personality changes, and impaired reasoning. The intellectual decline is usually progressive from mild cognitive impairment to dementia, the latter being a major health and socioeconomic problem of industrialized countries with high life expectancy.

Alzheimer's disease (AD) is the most common form of dementia. Typically diagnosed in patients over the age of 65, although an early onset form of the disease can strike much earlier. With the overall aging of the world's population, the prevalence of AD is expected to increase markedly. Other common forms of dementia include vascular dementia, Lewy body dementia, and frontotemporal dementia. Less common causes include normal pressure hydrocephalus, Parkinson's disease, syphilis, and Creutzfeld-Jakob disease among others. Other conditions of cognitive impairment include elderly with associated cognitive decline without known neurological disease and patients with cognitive decline associated with psychiatric disorder, autism, epilepsy, etc.

Typically, patients suffering from cognitive impairment experience good days during which they are lucid and capable of performing a variety of activities as well as bad days during which they feel confused, physically tired, and incapable of performing many even basic activities. The reason for these fluctuation as well as their origin is unknown in many cases. There thus remains a need for identifying and managing fluctuation in cognitive performance of subjects suffering from cognitive impairment.

Cognitive impairment may be also associated with consumption of many drugs such as psychiatric drugs, certain epilepsy drugs, statin and PPIs drugs, etc.

Cognitive impairment can also present as an acute event in the presence of a neuroglial disease in the background or in the absence of such. The cognitive decline can be temporary and resolve in a matter of days to weeks but in some cases it can last for months and even lead to permanent cognitive decline. A common example is delirium, which is a serious acute neuropsychiatric syndrome with core features of inattention and global cognitive dysfunction. Delirium is quite common in elderly hospitalized subjects but also present in the everyday surroundings in subjects that live in the community and can be triggered by non-neurological conditions such as infection, dehydration or metabolic imbalances, such as low sodium or low calcium, use of drugs, sleep deprivation, surgery or other medical procedures that include anesthesia, pain etc. Because of the many factors/conditions that can lead to delirium, its clinical and cognitive manifestation varies and is sometimes difficult to be detected early enough.

The foregoing examples of the related art and limitations related therewith are intended to be illustrative and not exclusive. Other limitations of the related art will become apparent to those of skill in the art upon a reading of the specification and a study of the figures.

### SUMMARY

The following embodiments and aspects thereof are described and illustrated in conjunction with systems, tools and methods which are meant to be exemplary and illustrative, not limiting in scope.

The systems and methods disclosed herein advantageously provide a thorough and comprehensive analysis to aid the patient's care giver and/or the patient himself to maximize his/her cognitive status and/or detect early signs of acute decline such as decline associated with delirium and optionally trigger an alarm preferably accompanied by physiological related information, either as presentation of abnormal parameters and/or as proposing the subject suffers from potential conditions such dehydration, infection, sleep disturbances, etc. In accordance with some embodiments, the system may also identify peaks in various cognitive functions in the elderly or patients with a chronic neurological disease and attempt to identify correlation with external factors to enable maximizing cognitive function and avoid falls.

The systems and methods disclosed herein advantageously offer accurate assessment of cognitive performance based on comprehensive analysis of determined cognitive performance based on and/or in response to a performed cognitive training and passively monitored cognitive function and life style, and/or physiological and/or medical parameters' monitoring. Advantageously, the parameters may be monitored frequently as part of “routine” training, while avoiding overload that would be caused, if dedicated testing sessions would be performed. As a further advantage, the parameters may be monitored in any location, such as at ease in a subject's home, and thus avoid a stress and bias associated with hospitalization or even a doctor's session, which may affect results of a cognitive training. Furthermore, the parameters may be monitored as part of the patient's routine and/or cognitive training, thus avoiding the overload caused when monitoring is performed at dedicated testing sessions.

According to some embodiments, there is provided a computer implemented method for managing a subject suffering from cognitive impairment, the method comprising: providing to a subject a cognitive training session; determining at least one aspect of the subject's cognitive performance based on and/or in response to the training session; monitoring one or more life style, physiological and/or medical parameters of the subject before, during and/or after the training session; identifying peaks (positive and negative) in the subject's cognitive performance; wherein the identifying comprises comparing the determined cognitive performance to stored cognitive performance data; wherein the stored cognitive performance data comprise cognitive performance test results of the subject obtained during previous cognitive training sessions and/or cognitive performance test results of other subjects suffering from a cognitive impairment and having at least one similar patient characteristic; identifying changes in one or more life style (such as food, food supplements, physical activity, etc.), physiological and/or medical parameters positively or negatively associated with the peak in the cognitive performance; and providing the subject with a life style, physiological and/or medical recommendation based on the identified association. The correlation can be with a phase shift. In order to identify a meaningful and/or significant correlation, data from other patients can be incorporated. Similarly, data from a monitored individual can help in understanding the correlation between certain life styles, physiological and/or medical parameters and cognitive performance in a relevant population.

In some embodiments, the other subjects are suffering from the same type of cognitive impairment as the trained subject.

In some embodiments, the cognitive impairment is associated with Alzheimer's disease.

In some embodiments, the method further comprises providing to the subject a second training session and determining the at least one aspect of the subject's cognitive performance based on and/or in response to said second training session.

In some embodiments, the method further comprises comparing the cognitive response obtained in response to said first and second training sessions and determining training characteristics associated with a better cognitive performance.

In some embodiments, the one or more training characteristics comprise type of training, performances of specific cognitive capabilities and ratio between them, length of training, frequency of training sessions, subject's compliance to the training sessions, or any combination thereof.

In some embodiments, the first and second training sessions are different.

In some embodiments, the one or more medical parameters are selected from the group consisting of: drug administered, medical treatment, blood and urine tests, physiotherapy, psychological treatment, psychiatric treatment, or any combination thereof.

In some embodiments, the one or more life style parameters are selected from the group consisting of: physical activity, nutrition, consumption of food supplements, social interactions, sleep quality, sleep/wakefulness, a degree of maintaining daily routine, or any combination thereof.

In some embodiments, the one or more physiologic parameters are selected from the group consisting of: body temperature, respiratory rate, pulse rate, blood pressure, blood glucose, blood insulin, blood oxygen, cholesterol, blood pH value, body fat, skin resistance, SpO₂, body temperature, respiratory effort, EEG, hepatic enzymes, blood count, sodium potassium electrolytes, or any combination thereof.

In some embodiments, the method further comprises the step of assigning a score representing the subject's cognitive status. In some embodiments different scores are given for different aspects of cognition, including memory, problem solving, confusion, alertness, attention, etc.

In some embodiments, the method further comprises the step of assigning a score representing the subject's cognitive status during adequate training while the subject undergoes an EEG test. This allows comparison of changes in EEG while the same stimulation is performed and correlate between EEG and cognitive function scores. In some embodiments, the EEG tests may be performed by consumer EEGs that enable convenient testing that can be done routinely without the workload required in placing a large number of electrodes. Examples of EEGs that may be used include, Emotiv Insight, Muse2016, Imec, etc. In some embodiments, different scores are given to different aspects of cognition, including memory, problem solving, confusion, alertness, attention, etc.

In some embodiments, the method further comprises identifying, based on said comparison, a deterioration in cognitive performance and providing the subject with a life style, physiological and/or medical recommendation based on the identification.

In some embodiments, the training is an active training comprising memory training, attention training, lingual training, numeric training, motoric training, social training, reading training, orientation training, problem solving, or any combination thereof.

In some embodiments, the training is passive training comprising daily activities such as conversation over the phone, voice analysis with/without content analysis, use of computer and/or cell phone, etc. that are monitored in order to identify peaks in cognitive performances or early signs of acute cognitive decompensation.

In some embodiments, a system triggers tasks at appropriate level and timing according to the patient/subject status to monitor his/her attention and detect early signs of an acute decline. An example can be sending a What's App message that the user needs to reply or promote to play a simple game. According to some embodiments, detecting early signs of acute decline may refer to a detection which is sufficiently early to enable preemptive measures to be taken.

In some embodiments, a patient at risk of an acute decline such as delirium undergoes evaluation when hospitalized and an appropriate training/monitoring protocol is selected that starts with a baseline cognitive evaluation. The motivation is to detect early signs of cognitive deterioration accompanied by acquisition and evaluation of physiological/biological data (from different sensors, use of drugs, blood and urine samples, etc.) from baseline data and continue with monitoring of biological related parameters to detect early signs of physiological deterioration that can lead to acute decline.

In some embodiments, the system generates alarms for acute conditions, such as indicating risk to develop events such as delirium (to enable preventing at least part of them) or early signs of such an event indicating that the patient may suffer from delirium, enabling early treatment in order to minimize complications and improve long-term outcome. The signs that lead to an alarm can be cognitive and/or physiological.

In some embodiments, the system generates alarms when acute conditions, such an acute cognitive decline (e.g. delirium) or early signs of such an event, are detected. According to some embodiments, the alarm is accompanied by an indication of a potential correlation between physiological triggers and changes in cognitive status. The biological information can be based on presentation of raw data indicating abnormalities and/or suggest potential conditions, such as infection, dehydration, etc. The interpretation can be based on analysis of data from an individual subject, using an algorithm that is developed based on analysis of data from other patients in order to provide a sensitive and reliable detection of changes, pointing on correlation with raw data and in some cases suggesting conditions. In some cases, in addition to analysis based on big data methodologies, the algorithm is fed with clinical flow charts and relevant medical wisdom.

In some embodiments, the method further comprises the step of monitoring every day activities performed by the subject.

In some embodiments, the method further comprises the step of identifying every day activities positively or negatively associated with the peak in the cognitive performance; and providing the subject with a recommendation based on the identified association.

According to some embodiments, there is provided a system for managing a cognitive training of a subject suffering from a cognitive impairment, the system comprising: a training unit configured to provide a cognitive training to the subject and to determine the subject's cognitive performance based on the training; one or more sensors configured to monitor one or more life style, physiologic and/or medical parameters of the subject before, during and/or after the training session; and a processing circuitry configured to identify peaks in the subject's cognitive performance; wherein the identifying comprises comparing the determined cognitive performance to stored cognitive performance data; wherein the stored cognitive performance data comprise cognitive performance test results of the subject obtained during previous cognitive training sessions and/or cognitive performance test results of other subjects suffering from a cognitive impairment and having at least one similar patient characteristic, the processing circuitry is further configured to: identify, based on the comparison, one or more life style, physiological and/or medical parameters positively or negatively associated with the peak in the cognitive performance; and provide the subject with a life style, physiological and/or medical recommendation based on the identified association.

According to some embodiments, there is provided a system for managing a cognitive condition of a subject suffering from cognitive impairment, the system comprising: a cognitive monitoring unit configured to monitor the subject and to determine a change in the subject's cognitive performance; one or more sensors configured to monitor one or more life style, physiologic and/or medical parameters of the subject before, during and/or after said monitoring period; and a processing circuitry configured to: identify peaks in the subject's cognitive performance; wherein the identifying comprises comparing the determined cognitive performance to stored cognitive performance data; wherein the stored cognitive performance data comprise cognitive performance test results of the subject obtained during previous cognitive monitoring periods and/or cognitive performance test results of other subjects suffering from cognitive impairment and having at least one similar patient characteristic, identify one or more changes in life style, physiological and/or medical parameters positively or negatively associated with the peak in the cognitive performance; and provide the subject with a life style, physiological and/or medical recommendation based on the identified association.

According to some embodiments, there is provided a computer implemented method for managing a cognitive condition of a subject suffering from cognitive impairment, the system comprising: monitoring the subject and determining a change in the subject's cognitive performance; monitoring one or more life style, physiologic and/or medical parameters of the subject before, during and/or after said monitoring period; identifying peaks in the subject's cognitive performance; wherein the identifying comprises comparing the determined cognitive performance to stored cognitive performance data; wherein the stored cognitive performance data comprise cognitive performance test results of the subject obtained during previous cognitive monitoring periods and/or cognitive performance test results of other subjects suffering from cognitive impairment and having at least one similar patient characteristic, identifying one or more changes in life style, physiological and/or medical parameters positively or negatively associated with the peak in the cognitive performance; and providing the subject with a life style, physiological and/or medical recommendation based on the identified association.

According to some embodiments, there is provided a computer implemented method to prevent/avoid or minimize cognitive drops, the method includes: providing to a subject a cognitive monitoring tool, the cognitive monitoring tool comprising at least one passive monitoring and/or training session; determining at least one aspect of the subject's cognitive performance based at least on the cognitive monitoring tool; monitoring one or more life style, physiological and/or medical parameters of the subject before, during and/or after the passive monitoring and/or training session; identifying a decline in the subject's cognitive performance; wherein the identifying comprises: comparing the determined cognitive performance to stored cognitive performance data; wherein the stored cognitive performance data comprise cognitive performance test results of the subject obtained during previous cognitive sessions; identifying one or more physiological and/or medical parameters positively or negatively associated with the decline in the cognitive performance; and providing an output signal indicative of one or more physiological and/or medical parameters positively or negatively associated with the decline in the cognitive performance.

The method may further include providing a physiological and/or medical recommendation based on the identified association.

According to some embodiments, the other subjects are suffering from the same cognitive impairment as the monitored subject.

According to some embodiments, the one or more physiologic parameters are selected from the group consisting of: body temperature, respiratory rate, SpO2, respiration effort, pulse rate, blood pressure, blood sugar, blood oxygen, blood pH level, cholesterol or other blood/urine test parameters, body fat, skin resistance, or any combination thereof.

According to some embodiments, the one or more physiologic parameters are selected from the group consisting of: EEG signal, ECG signal, movement sensors and any combination thereof.

According to some embodiments, the one or more medical parameters are selected from the group consisting of: drug administered, medical treatment, physiotherapy, psychological treatment, psychiatric treatment, or any combination thereof.

According to some embodiments, the one or more life style parameters are selected from the group consisting of: physical activity, nutrition (food and liquids), consumption of food supplements, social interactions, sleep quality, sleep/wakefulness, degree of maintaining daily routine, or any combination thereof.

According to some embodiments, the processing circuitry is further configured to and/or the method further includes assigning at least one score representing the subject's cognitive status.

According to some embodiments, the processing circuitry is further configured to and/or the method further includes identifying, based on said comparison, a deterioration in cognitive performance and to provide the subject with a life style, physiological and/or medical recommendation based on the identification.

According to some embodiments, the system/method further includes a training unit configured to provide cognitive training to the subject and to determine the subject's cognitive performance based on said training.

According to some embodiments, the training is an active training comprising memory training, attention training, lingual training, numeric training, motoric training, social training, reading training, orientation training, problem solving, or any combination thereof.

According to some embodiments, the processing circuitry is further configured to and/or the method further includes identifying every day activities positively or negatively associated with the peak in the cognitive performance; and providing the subject with a recommendation based on the identified association.

According to some embodiments, the training includes memory training, attention training, lingual training, numeric training, motoric training, social training, reading training, orientation training, or any combination thereof.

According to some embodiments, the training unit comprises a user interface for input and/or output, said user interface selected from a group consisting of video, cellular, computer based, audio, tactile interface, or any combination thereof.

According to some embodiments, the processing circuitry is operably linked to a data storage unit for storing said stored cognitive performance data. According to some embodiments, the data storage unit is cloud based.

According to some embodiments, the system/method may further include a learning module configured to learn correlation of different (cloud based) cognitive scores, physiological parameters and combination thereof and use the correlations to identify correlation in subject.

According to some embodiments, the system/method may further include a cloud based learning module configured to provide prediction of delirium based on cognitive scores, physiological parameters, subject's data, or any combination combination thereof. Such prediction of delirium may be provided before correlation is identified in the subject.

According to some embodiments, the system/method may further include interface with hospital/Clinics/HMO and/or other EMR/EHR system to access data about individual subject/patients to be used to look for changes and correlations, and/or provide alarms.

According to some embodiments, the system/method may further interface with a clinical decision support system (CDSS). The system may get information/rules and provide such to the CCSS.

According to some embodiments, the (cloud-based) algorithms learn how to provide signals (such as alarms) of delirium before clear correlation is identified in the subject, based on big data analysis and medical know-how. According to some embodiments, this requires that data is sent to the cloud to improve sensitivity and specificity of alarms. According to some embodiments, the system/method provides such alarm signals.

According to some embodiments, the system/method may further include a cloud-based learning module configured to learn general cognitive scores, physiological parameters and combinations thereof from the data obtained from the subject.

According to some embodiments, the system/method may further include (a module for) recording and/or storing the subject's memories during periods of peak performance. Recording and/or storing may include tagging the memories to enable future access/retrieval.

In some embodiments, the other subjects are suffering from the same cognitive impairment as the trained subject.

In some embodiments, the cognitive impairment is associated with Alzheimer's disease.

In some embodiments, the cognitive impairment is associated with Parkinson disease.

In some the cognitive impairment is associated with aging and/or vascular diseases.

In some embodiments, the cognitive impairment is associated with psychiatric disease.

In some embodiments, the cognitive impairment is associated with “natural” decline associated with aging without known neurological diseases.

In some embodiments, the one or more physiologic parameters are selected from the group consisting of: body temperature, respiratory rate, pulse rate, blood pressure, blood glucose, blood oxygen, blood count, white cells, UTI, anemia parameters, electrolytes (sodium potassium), creatinine, PH arterial, liver enzymes, PaO2/SpO2, cholesterol, skin resistance, EEG, Polysomnography or any of the following parameters , eye movements (EOG), muscle activity or skeletal muscle activation (EMG), and heart rhythm (ECG), anemia tests, or any combination thereof.

In some embodiments, aiming to avoid delirium and/or to facilitate early detection of delirium may include integrating specific biomarkers such as, but not limited to, those proposed in:

i. Biomarkers for delirium—A Review; Khan et al. J Am Geriatr Soc. 2011 November.

ii. Interrelationship Between Delirium and Dementia Review Article Serum Biomarkers for Delirium; Edward R. Marcantonio et al. Journal of Gerontology 2006.

iii. Delirium—biomarkers and genetic variance; Nicoleta Stoicea et al. Frontiers in Pharmacology 2014.

In some embodiments, the one or more medical parameters are selected from the group consisting of: drug administered, medical treatment, physiotherapy, psychological treatment, psychiatric treatment, or any combination thereof.

In some embodiments, the one or more life style parameters are selected from the group consisting of: physical activity, nutrition, consumption of food supplements, social interactions, sleep quality, sleep/wakefulness, a degree of maintaining daily routine, or any combination thereof.

In some embodiments, the processing circuitry is further configured to assign at least one score representing the subject's cognitive status.

In some embodiments, the processing circuitry is further configured to identify, based on the comparison, a deterioration in cognitive performance and to provide the subject with a life style, physiological and/or medical recommendation based on the identification.

In some embodiments, the training is an active training comprising memory training, attention training, lingual training, numeric training, motoric training, social training, reading training, orientation training, problem solving, or any combination thereof.

In some embodiments, the training unit is further configured to monitor every day activities performed by the subject.

In some embodiments, the processing circuitry is further configured to identify every day activities positively or negatively associated with the peak in the cognitive performance; and providing the subject with a recommendation based on the identified association.

In some embodiments, the training unit is configured to provide memory training, attention training, lingual training, numeric training, motoric training, social training, reading training, orientation training, or any combination thereof.

In some embodiments, the training unit comprises a user interface for input and/or output, the user interface selected from a group consisting video, cellular, computer based, audio, tactile interface, or any combination thereof.

In some embodiments, the processing circuitry is operably linked to a data storage unit for storing the stored cognitive performance data. In some embodiments, the data storage unit is cloud based.

In some embodiments, the system further comprises a learning module configured to learn correlation of different cloud based cognitive scores, physiological parameters and combination thereof and use the correlations to identify correlation in subject.

In some embodiments, detection of early signs of acute conditions such as delirium, is based, in addition to the individualized cognitive a biological/physiological information, also on data obtained from other patients that is analyzed in the cloud to improve a detection algorithm.

In some embodiments, a data base in the cloud is fed with retrospective data to optimize an algorithm to detect signs of acute events with high sensitivity and specificity, i.e., the cloud is fed with clinical, cognitive and physiological data from patients/subjects, including cases wherein the system did not find/identify a correlation in real time or that were not monitored by the system in order to improve an algorithm to find a correlation as identification of potential delays before changes in physiological score, change of medicine, etc. and the presentation of change in cognition score. The algorithm can be continuously improved by analysis of new data as it is gathered using data mining methodologies, including statistical methods and/or combined with medical know-how.

In some embodiments, detection of fluctuations in cognition and/or acute falls in cognition, is based, in addition to the individualized cognitive and biological/physiological information, also on data obtained from other patients that is analyzed in the cloud to improve the analysis and detection algorithm. An algorithm can be developed based on big data, data mining methodologies, etc.

In some embodiments, the training is used to download and upload an individual's memories not just for the purpose of learning/practicing but to enable memory refresh and upload of memories if and when there is a memory decline. This is another example of the present invention wherein training is used beyond its obvious purpose in order to improve management of cognitive disorders and improve the well-being of individuals with cognitive disorders.

In some embodiments, memories include multiple tags which are uploaded (saved in individual computers and/or memory modules and/or the cloud) that can facilitate their retrieval.

In some embodiments multiple tags include names, images, associated links, songs and other sensing tags.

In some embodiments, the system further comprises a cloud based learning module configured to learn general cognitive scores, physiological parameters and combination thereof from the data obtained from the subject.

More details and features of the current invention and its embodiments may be found in the description and the attached drawings.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE FIGURES

Exemplary embodiments are illustrated in referenced figures. Dimensions of components and features shown in the figures are generally chosen for convenience and clarity of presentation and are not necessarily shown to scale. It is intended that the embodiments and figures disclosed herein are to be considered illustrative rather than restrictive. The figures are listed below:

FIG. 1 is a block diagram of a system for managing a subject's cognitive training of a subject suffering from a cognitive impairment or in risk to develop same, according to an exemplary embodiment of the current disclosure;

FIG. 2 is a flow chart of the steps of a method for managing a subject suffering from a cognitive impairment or at risk of developing same, according to an exemplary embodiment of the current disclosure;

FIG. 3 is a flow chart of the steps of a method for managing patients at risk of acute cognitive deterioration, for providing alarms and for identifying potential causes and/or abnormal physiological parameters associated with the acute cognitive deterioration; and

FIG. 4 is a high-level overview flow chart of an entire system for minimizing/avoiding cognitive drops in acute (delirium) and/or chronic cognitive impairment indications.

### DETAILED DESCRIPTION

Disclosed herein are systems and methods for minimizing cognitive falls by detecting fluctuation in cognitive performance in a subject suffering from a cognitive impairment (e.g., dementia associated with aging and/or known neurological disease) or avoiding/minimizing acute cognitive falls in patients at risk based on monitoring of cognitive performance and physiologic parameters before, during, and/or following the cognitive training/passive cognitive monitoring and detection of correlation between changes in cognitive scores and changes in physiological related parameters.

Advantageously, the disclosed systems and methods are used for identifying peaks and/or deterioration in cognitive performance of a subject suffering from cognitive impairment, and provide the subject with a training/physiological recommendation based on one or more physiological and/or training characteristics identified as being associated with the peak (positive/elevated performances) and/or deterioration of obtainable cognitive performance.

Advantageously, the disclosed systems and methods may be used for identifying early signs of acute deterioration in cognitive performance of a subject suffering from cognitive impairment or subjects that do not suffer from significant chronic cognitive impairment, but are at risk to develop such impairment. One example is patients who are prescribed with new drugs, such as psychoactive, antifungal, statins, blood pressure and glucose control drugs that may lead to cognitive deterioration. According to some embodiments, the system may be further configured to serve as, to provide or to trigger a tool to avoid delirium in elderly patients that are at risk to develop such a condition for example, but not necessarily, when hospitalized. The objective is to alarm the subject and/or a care-giver of the deterioration and, when applicable, to provide a physiological recommendation based on one or more physiological and/or cognitive characteristics identified as being associated with the acute event or with the risk of developing a severe cognitive event (such as, but not limited to, delirium) in the short term. It is therefore the objective to detect signs of conditions such as delirium early enough to provide the subject with treatment as early as possible and/or to prevent such events from occurring.

The system may provide a thorough and comprehensive analysis to aid the patient's caregiver (e.g., a medical doctor). Optionally, a list of recommended life style, physiological and medical recommendations may be provided. These recommendations may be evaluated by a patient's caregiver. Once the caregiver either signs off on the recommended training and/or physiological recommendation or makes adjustments (which are also recorded and noted for future use), the adjusted training/physiological recommendation may be applied.

Advantageously, during training, the system may learn a correlation between cognitive function and applied training sessions, life style, physiological and/or medical parameters, on an individual level or on a community-based level.

The term “cognitive impairment” as used herein relates to a condition which can be characterized as a loss, usually progressive, of cognitive and intellectual functions characterized by disorientation, impaired memory, judgment and intellect and a shallow labile affect. The impairment may be caused by a variety of disorders including severe infections and toxins, but most commonly associated with structural brain disease. According to some embodiments, the cognitive impairment may be dementia, including, but not limited to, AIDS dementia, Alzheimer dementia, pre-senile dementia, senile dementia, catatonic dementia, dialysis dementia (dialysis encephalopathy syndrome), epileptic dementia, hebephrenic dementia, Lewy body dementia (diffuse Lewy body disease), multi-infarct dementia (vascular dementia), paralytic dementia, posttraumatic dementia, dementia praecox, primary dementia, toxic dementia and vascular dementia.

As used herein, the term “cognitive function” refers to the special, normal, or proper physiologic activity of the brain, including one or more of the following: mental stability, memory/recall abilities, problem solving abilities, reasoning abilities, thinking abilities, judging abilities, ability to discriminate or make choices, capacity for learning, ease of learning, perception, intuition, attention, alertness, response time to stimulation and awareness.

As used herein, the terms “disease” or “disorder” refer to an impairment of health or a condition of abnormal functioning.

As used herein, the term “subject” refers to any animal, including, but not limited to, humans and non-humans. Typically, the terms “patient” and “subject” are used interchangeably herein. Optionally, the subject is a human subject. The subject suffering from a cognitive impairment may be a dementia associated with aging patient. Optionally, the cognitive impairment is associated with Alzheimer's disease or other neurological disease.

According to some embodiments, there is provided a method/system for managing a subject suffering from cognitive impairment. The method/system includes determining at least one aspect of the subject's cognitive performance based on and/or in response to a training provided to the subject. The training may be performed in the context of a computer-based cognitive training exercise. The training may preferably be provided in a repeated manner such as once a day, twice a day, once every two days, once a week, bi-weekly, once a month or any other suitable amount of time suitable for efficient evaluation and/or monitoring of subjects suffering from cognitive impairment. The method/system further includes identifying peaks and/or deterioration in the subject's cognitive performance by comparing the determined cognitive performance to stored cognitive performance data; wherein the stored cognitive performance data include cognitive performance test results of the subject obtained during previous cognitive training sessions and/or cognitive performance test results of other subjects suffering from cognitive impairment and having at least one similar patient characteristic. The method/system further includes comprehensive monitoring of life style, physiological and/or medical parameters of the patient using one or more sensors, such as 1, 2, 3, 4, 5 or more sensors. Each possibility is a separate embodiment. The monitoring may be done before, during and/or after training. According to some embodiments, at least one of the parameters (e.g. behavioral parameters) may be recorded using a user interface. Alternatively, all parameters, including behavioral parameters, may be recorded in a patient independent manner, for example, including video monitoring of the patient. Based on the monitored parameters, life style, physiological and/or medical parameters positively or negatively associated with the peak in the cognitive performance may be identified. According to some embodiments, the identification of associated parameters may further be based on data sets of monitored parameters obtained for the subject during previous trainings and/or data sets of monitored parameters obtained from other patients suffering from cognitive impairment and sharing at least one patient characteristic with the evaluated subject. Once associated life style parameters, physiological parameters and/or medical parameters are identified, a life style, physiological and/or medical recommendation may be provided to avoid falls in cognition and/or maximize the duration of the high performance, thereby prolonging and/or increasing the frequency of positive peaks in cognitive performance. As used herein, the term “prolonging” a peak in cognitive performance may include increasing the length of the peak by at least 5%, at least 10% or at least 15%. Each possibility is a separate embodiment. As used herein, the term “increasing the frequency” of peaks in cognitive performance may include increasing the number of peaks by at least 5%, at least 10% or at least 15%. Each possibility is a separate embodiment.

According to some embodiments, the identification of associated parameters and correlation between changes in different scores of cognition function and physiological parameters may be based on artificial intelligence methodologies and/or machine learning techniques such as “deep learning”, which techniques are known in the art. According to some embodiments, the machine learning techniques may “learn” the correlation between cognitive performance and physiology, life style and/or medication on an individual and/or community-based level. According to some embodiments, the learning may be performed while training, i.e. each training session may be incorporated into the learning module so as to further adjust and/or improve the algorithm. Similarly, data mining techniques may be used to identify patterns in large population data bases using Bayesian statistics in a non-trivial manner, for example. This can allow the system to identify patterns of correlations between physiological changes and different cognitive functions and increase sensitivity and accuracy of identifications and predictions on an individual basis. As an example, longitudinal cognitive data and physiological data from a patient at risk of developing delirium can be collected and retrospectively analyzed to detect patterns that enable prediction of development of delirium, early signs of delirium and/or identification of the underlying cause of delirium vs. patterns that are not associated with development of delirium even in patients at risk.

Machine learning and data mining techniques are known in the art, therefore the details are not described herein, however, a few reviews are fully incorporated herein:
I. Deo R C. Machine Learning in Medicine. Circulation. 2015; 132(20):1920-1930. doi:10.1161/CIRCULATIONAHA.115.001593. [https://www.ncbi.nlm.nih.gov/pmc/articles/PMC5831252/]
II. S. B. Kotsiantis Informatica 31 (2007) 249-268 249 [https://datajobs.com/data-science-repo/Supervised-Learning-[SB-Kotsiantis].pdf]
III. Omar Y. Al-Jarrah et al, Big Data Research Volume 2, Issue 3, September 2015, Pages 87-93
IV. Hian Chye Koh et al, Journal of Healthcare Information Management—Vol. 19, No. 2
V. Matthew Herland, et al, Journal of Big Data20141:2
VI. https://en.wikipedia.org/wiki/Machine_learning and https://en.wikipedia.org/wiki/Data_mining

The interface used (in one or two ways) to manage the patients/subjects may be any suitable interface such as, but not limited to, clinical decision support system (CDSS) and may provide rules for management in particular. CDSS are known in the art so the details are not described herein, however, a few reviews are fully incorporated herein:
I. https://en.wikipedia.org/wiki/Clinical_decision_support_system
II. Prabhhu Murugesan et al CSC 2014 http://assets1.csc.com/innovation/downloads/Clinical_Decision_Support_Systems.pdf
III. MARK A. MUSEN, et al, Clinical Decision-Support Systems http://eknygos.Ismuni.It/springer/56/698-736.pdf,

Similarly, the system may be an interface with HER/EMR systems.

Non-limiting examples of shared patient characteristics include the same type of cognitive impairment, the same age range, the same body mass, and the same gender. The other subjects may be suffering from the same type of cognitive impairment as the trained subject. Further, the cognitive impairment of both the other subjects and the trained subject may be at the same severity/stage. Optionally, the cognitive impairment is associated with a disease selected from the group consisting of: AIDS dementia, Alzheimer dementia, pre-senile dementia, senile dementia, catatonic dementia, dialysis dementia (dialysis encephalopathy syndrome), epileptic dementia, hebephrenic dementia, Lewy body dementia, multi-infarct dementia (vascular dementia), paralytic dementia, posttraumatic dementia, dementia praecox, primary dementia, toxic dementia and vascular dementia. Optionally, the cognitive impairment is associated with Alzheimer's disease.

Optionally, the subject's memories are recorded when a peak in cognitive performance is identified. Optionally, at least one of a subject's training characteristics, physiological, medical and lifestyle parameters are stored, such as in a data storage unit, at least when a peak, or a deterioration in cognitive performance is identified.

Optionally, the subject is provided with a second training session, and determining at least one aspect of the subject's cognitive performance based on and/or in response to the second training session. The first and second training sessions may have the same training characteristics. Alternatively, the second training session may differ by one or more characteristics from the first training session.

The training characteristics of the first and the second training sessions may include type of training, performances of specific cognitive capabilities and ratio between them, length of training, frequency of training sessions, subject's compliance to the training sessions, or any combination thereof. Non-limiting examples of training types include memory training, attention training, lingual training, numeric training, motoric training, social training, reading training, orientation training, problem solving, or any combination thereof. The training sessions may be conducted periodically (e.g., each day, once a week, etc.). Each of the training sessions may be conducted for a duration ranging of 0.1 to 1 minute, 0.1 to 2 minutes, 0.1 to 3 minutes, 0.1 to 4 minutes, 0.1 to 5 minutes, 0.1 to 6 minutes, 0.1 to 7 minutes, 0.1 to 8 minutes, 0.1 to 9 minutes, 0.1 to 10 minutes, 0.1 to 20 minutes, 0.1 to 30 minutes, 0.1 to 40 minutes, 0.1 to 50 minutes, 0.1 to 60 minutes, 0.1 to 90 minutes, 0.1 to 120 minutes, 0.1 to 180 minutes, 0.1 to 240 minutes, 0.5 to 1 minute, 0.5 to 2 minutes, 0.5 to 3 minutes, 0.5 to 4 minutes, 0.5 to 5 minutes, 0.5 to 6 minutes, 0.5 to 7 minutes, 0.5 to 8 minutes, 0.5 to 9 minutes, 0.5 to 10 minutes, 0.5 to 20 minutes, 0.5 to 30 minutes, 0.5 to 40 minutes, 0.5 to 50 minutes, 0.5 to 60 minutes, 0.5 to 90 minutes, 0.5 to 120 minutes, 0.5 to 180 minutes, 0.5 to 240 minutes, 1 to 2 minutes, 1 to 3 minutes, 1 to 4 minutes,1 to 5 minutes, 1 to 6 minutes, 1 to 7 minutes, 1 to 8 minutes, 1 to 9 minutes, 1 to 10 minutes, 1 to 20 minutes, 1 to 30 minutes, 1 to 40 minutes, 1 to 50 minutes, 1 to 60 minutes, 1 to 90 minutes, 1 to 120 minutes, 1 to 180 minutes, 1 to 240 minutes, 5 to 6 minutes, 5 to 7 minutes, 5 to 8 minutes, 5 to 9 minutes, 5 to 10 minutes, 5 to 20 minutes, 5 to 30 minutes, 5 to 40 minutes, 5 to 50 minutes, 5 to 60 minutes, 5 to 90 minutes, 5 to 120 minutes, 5 to 180 minutes, 5 to 240 minutes, 10 to 20 minutes, 10 to 30 minutes, 10 to 40 minutes, 10 to 50 minutes, 10 to 60 minutes, 10 to 90 minutes, 10 to 120 minutes, 10 to 180 minutes, 10 to 240 minutes. Each possibility represents a separate embodiment of the present disclosure.

The cognitive response obtained in response to the first and second training sessions is compared. In a non-limiting example, the cognitive response may include quantification of physical reaction time; perceptual awareness thresholds; brain-speed, degree of focus/attention; and the speed, efficiency and capacity of elementary cognitive processes, including choice, discrimination and decision responses, memory-access and information-retrieval. Optionally, a score representing the subject's cognitive status is assigned based on at least one of the measured cognitive responses. Optionally, the score is assigned based on a summary of a plurality of the measured cognitive responses. Optionally, the comparison is done by comparing the assigned cognitive scores of the first and second training session. Optionally, training characteristics associated with a better cognitive performance are determined and training is optimized.

When a deterioration or improvement in cognitive performance is identified, based on the comparison of the cognitive responses obtained in response to the first and second training sessions, the subject is provided with a life style, physiological and/or medical recommendation based on the identification.

The lifestyle, the physiological, and the medical parameters monitored may be further indicative of or assist in assessing cognitive performance. Such measurements may include vital signs, sleep patterns, movement and exercise patterns, dietary habits, glucose levels, drug consumption and so on (such devices are known to people pursuing the field of “quantified self”). Suitable types of sensors for monitoring the lifestyle, the physiological, and/or the medical parameters include, but are not limited to: electrodes, LED Emitter and optical sensor configured for example to measure blood volume pulse detection sensor, strain gauge configured for example to measure change in chest volume which is indicative of respiration rate, thermistors configured for example to measure skin temperature, thermopile configured for example to measure heat flux, a thin film piezoelectric sensor configured for example to measure eye movement, a Sphygmomanometer configured for example to measure blood pressure, an electro-chemical sensor configured for example to measure oxygen consumption, blood glucose sensor configured for example to glucose level, accelerometer configured for example to measure body movement indicative of activity, mercury switch array configured for example to measure body position (e.g., supine, erect, sitting).

The physiological parameters may include blood and urine tests, heart rate, pulse rate, beat-to-beat heart variability, pulse transit time, ECG, respiration rate, respiration effort, skin temperature, core body temperature, heat flow of the body, SPO₂, pulse transit time, sleep monitoring, galvanic skin response (GSR), electromyography (EMG), electroencephalogram (EEG), electrooculography (EOG), blood pressure, body fat, hydration level, blood sugar level, pressure on muscles or bones, and UV radiation exposure and absorption. Optionally, the one or more physiologic parameters are selected from the group consisting of: body temperature, respiratory rate, pulse rate, blood pressure, blood sugar, blood oxygen, blood pH level, blood electrolytes, cholesterol, body fat, skin resistance, urine tests, or any combination thereof.

Information relating to a patient's physiological state may be derived based on the data indicative of the measured physiological parameters. In a non-limiting example, stress/relaxation level may be determined based on parameters, such as EKG, beat-to-beat variability (and HRV), heart rate, pulse rate, respiration rate, skin temperature, heat flow, galvanic skin response, PaO₂/SpO₂, body temperature, EMG, EEG, blood pressure, activity, and oxygen consumption.

In certain cases, the data indicative of the various physiological parameters is the signal or signals themselves generated by the one or more sensors and, in certain other cases, the data is calculated by a processor based on the signal or signals generated by the one or more sensors. Methods for generating data indicative of various physiological parameters and sensors to be used therefor are well known in the art. In a non-limiting example, heart rate may be determined by electrocardiogram (ECG) which utilizes two electrodes (the sensors) to measure direct current which is further processed by a processor. In another non-limiting example, muscle pressure is measured by thin film piezoelectric sensors, and change in direct current is measured and processed. In another non-limiting example, skin conductance is measured by two electrodes, and the direct current is used to determine the galvanic skin response. In another non-limiting example, in order to determine respiration rate a change in chest volume is determined by utilizing a strain gauge sensor which generates a signal of change in resistance which is further processed.

Optionally, the one or more medical parameters are selected from the group consisting of: recent blood and urine tests and trend of changes therein, drugs zo administered, medical treatment, physiotherapy, psychological treatment, psychiatric treatment, or any combination thereof. These parameters may be registered or loaded into a processor.

Optionally, the one or more life style parameters are selected from the group consisting of: physical activity, nutrition, consumption of food supplements, social interactions, sleep quality, sleep/wakefulness, a degree of maintaining daily routine, or any combination thereof. Lifestyle parameters may be registered or loaded into a processor, directly measured, and alternatively or additionally, determined according to measured physiological parameters. According to some embodiments, the lifestyle parameters may be measured based on an analysis of video recording of the subject. In a non-limiting example, physical activity may be determined based on measured physiological parameters such as heart rate, pulse rate, respiration rate, heat flow, activity, and oxygen consumption. In another non-limiting example, sleep/wakefulness may be determined based on measured physiological parameters such as Beat-to-beat variability, heart rate, pulse rate, respiration rate, skin temperature, core temperature, heat flow, galvanic skin response, EMG, EEG, EOG, blood pressure, and oxygen consumption.

Additionally, the one or more sensors may also generate data indicative of various contextual parameters relating to the environment surrounding the patient. In a non-limiting example, the one or more sensor generate data indicative of the air quality, sound level/quality, light quality and/or ambient temperature near the patient, or even the global positioning of the patient. The one or more sensors may generate signals in response to contextual characteristics relating to the environment surrounding the individual, the signals ultimately being used to generate the type of data described above. Such sensors are well known, as are methods for generating contextual parametric data such as air quality, sound level/quality, ambient temperature and global positioning.

Everyday activities such as walking, eating, etc., performed by the subject, may be further monitored. According to some embodiments, the everyday activities may be monitored for example by video monitoring. Optionally, the method may further provide identification of everyday activities positively or negatively associated with the peak in the cognitive performance; and providing the subject with a recommendation based on the identified association.

In some embodiments, the method/system may further include recording or otherwise storing the subject's memories during periods of peak performance. According to some embodiments, the memories may be tagged to enable future access/retrieval for example during periods of non-peak performance. According to some embodiments, the tagging may be visual, i.e. icons and/or images associated with the memory, additionally or alternatively the tagging may be verbal, such as, but not limited to, a short sentence associated with the memory. It is understood that other ways of tagging may also be applicable and thus within the scope of this disclosure.

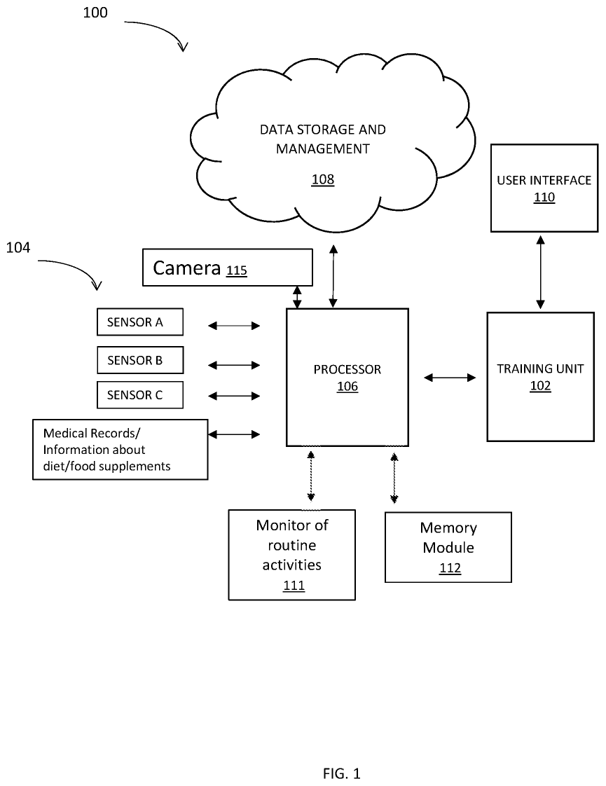

Reference is now made to FIG. 1, which shows a block diagram of a system 100 that may be used for a system for managing a subject suffering from a cognitive disorder or at a risk of developing same, in accordance with an embodiment.

System 100 includes a training unit 102 configured to provide to a subject a cognitive training session and to determine the subject's cognitive performance based on the training session; one or more sensors 104, denoted by way of example as SENSOR A, SENSOR B, SENSOR C, SENSOR D, configured to monitor one or more life style, physiological and/or medical parameters of the subject before, during and/or after the training session; and a processing circuitry 106 configured to determine the subject's cognitive status based on the tested cognitive performance and life style, physiological and/or medical parameters associated with peak performance and provide the subject with a life style, physiological and/or medical recommendation based on parameters identified as being associated with peak performance.

Training unit 102 may provide and monitor a memory training, attention training, lingual training, numeric training, motoric training, social training, reading training, orientation training, or any combination thereof. Training unit 102 and/or sensors 104 may further be configured to monitor passive training comprising everyday ordinary activities performed by the subject.

Optionally, training unit 102 includes a user interface 110 for input and/or output. Suitable user interfaces are selected from the group consisting of: video, cellular, computer-based, audio, tactile interface, or any combination thereof. Optionally, user interface 110 may display data received from processor 106 such as training/physiological recommendation(s). The display may be in a form of graphics, text, and other data.

Optionally, a monitoring unit 111 is used to monitor cognitive and psychological performances in a manner that does not require cooperation from the subject. Such use may be termed passive/routine monitoring and monitoring unit 111 may be termed a passive/routine monitoring unit. Examples of means to monitor cognitive/psychological performances based on monitoring of routine activities include analysis of voice patterns (clarity of pronunciation, loudness, wealth of vocabulary and content), texting in cell phone applications, communication with others (length, number, etc.), analysis of use of internet, analysis of calendar, etc.

Optionally system 100 may include a memory module 112 that is used to save memories with their tags. The module can be in a personal computer/cell phone, external HD, etc. and/or in the cloud. The upload and download are managed by processor 106.

The physiological parameters monitored by one or more sensors 104 may be indicative of or assist in assessing cognitive performance. Any monitoring wearable device capable of detecting or determining one or more data sets that may be utilized by the one or more methods and systems disclosed herein may be provided. Optionally, the one or more physiologic parameters are selected from the group consisting of: body temperature, respiratory rate, pulse rate, blood pressure, blood sugar, blood to oxygen, blood pH level, cholesterol, body fat, skin resistance, drug administered, medical treatment, nutrition, food supplement, physical activity, or any combination thereof.

The identification of peaks in the subject's cognitive performance by processing circuitry 106 may be achieved by comparing the determined cognitive performance to stored cognitive performance data.

Optionally, the stored cognitive performance data includes cognitive performance test results of the subject which were obtained during previous cognitive training sessions. Alternatively and/or additionally, the stored cognitive performance data comprise cognitive performance test results of other subjects suffering from a cognitive impairment and having at least one similar patient characteristic. Optionally, the other subjects are suffering from the same cognitive impairment as the trained subject.

Optionally, processor 106 is further configured to identify one or more life style, physiological and/or medical parameters positively or negatively associated with the peak in the cognitive performance; and provide the subject with a life style, physiological and/or medical recommendation based on the identified association. In such cases the determination of the subject's cognitive status may be based on comparison of the tested cognitive performance and the one or more monitored physiologic parameters to stored cognitive performance data and stored physiologic parameters.

Optionally, processing circuitry 106 is further configured to assign one or more scores representative of the subject's cognitive status. Non-limiting examples of parameters characterizing a subject's cognitive status include memory, problem solving, orientation, etc. Optionally, processing circuitry 106 is further configured to assign at least one score representing the subject's cognitive status. Processing circuitry 106 may further be configured to identify, based on the comparison, a deterioration in cognitive performance obtainable for the subject and to provide the subject with a training and/or physiological recommendation based on the one or more identified training characteristics and/or physiologic parameters.

Optionally, processing circuitry 106 is operably linked to a data storage unit 108 for storing stored cognitive performance data and/or physiologic parameters. Suitable data storage unit includes, but are not limited to, a cloud based storage. Optionally, at least some aspects of a patient's characteristics are stored/recorded in data storage unit 108. Optionally, the patient's characteristics may include the demographic information (e.g., age, gender), type of cognitive impairment-related diseases and degree thereof, medical history such as a medication list, medication allergies, immunizations and vaccinations, and/or a list of past medical procedures, current therapy plan such as talk therapy, behavioral therapy, specific cognitive training, chemical therapy (e.g., pharmacotherapy, such as, the utilization of drugs), mechanical therapy such as electroconvulsive therapies (ECT), nutrition therapy, or any combination thereof. Further, a family medical history may be important if there is any family history of cognitive impairment-related disease, or of other symptoms or diseases that may complicate recommended therapies. Further information may include electroencephalography (EEG) recording, bio-specimen test such as, but not limited to, blood work, cerebrospinal fluid (CSF) results, and urine test results.

According to an alternative or additional embodiments, for cognitive training the subject may be provided with tools that enable cognitive monitoring in a passive mode such as based on voice analysis, analysis of texting, etc. such as, but not limited to, cellphone and/or application that runs on a cell phone and/or application that enables access to data on cell phone or other applications that run on a cell phone. Examples of such applications are such that perform texting analysis on SMS/WhatsApp message. Analysis of content/quality of audio data may be performed on-line or in the cloud. Examples include IBM Watson Tone Analysis, Ludwig voice analysis, WinterLight Labs and other technologies for automatic speech recognition, such as: https://www.researchgate.net/publication/281089548_Automatic_Detection_of_Mild_Cognitive_Impairment_from_Spontaneous_Speech_using_ASR described in: https://www.technologyreview.com/s/603200/voice-analysis-tech-could-diagnose-disease/). Other examples are sensors that monitor movement to monitor rate and stability of movement.

For illustrative purposes only, a simplified example of a function that assesses correlation between a specific cognitive score of problem solving [PS(t) derived from a training game, from baseline measurement and x days afterwards (can be derived from multiple points) and some physiological changes [Blood Pressure, BP(t), and Medication to Change, MC(t) is:

PS(t=0)−PA(t=x) vs. [BP(t=0)−BP(t=x)]and/or MC(t=x−alpha)

Alpha—is a parameter that reflects expected delay between change in medicine and potential impact on cognition. While the algorithm may be fed with initial values for specific drugs this can be adjusted and optimized with time based on learning on a large population.

A similar example in looking for a correlation between changes in alertness following a patient's admittance to hospitalization to undergo surgery and alertness is evaluated at baseline t=0 and several physiological parameters as body temperature (BT), EEG based score

Alert(t=0)−Alert(t=y) vs. [BT(t=y)−BT(y−24)] OR BT(t=y)−BT(t=0)] and/or EEG(t=0)−EEG(y) and/or BC(0)−BC(y−beta)

BC(t)—Complete blood cell count with differential (can be helpful to diagnose infection and anemia)

Beta—is a parameter that reflects expected delay between signs of infection in blood count and potential impact on cognition. While the algorithm may be fed with initial values for specific infections and patients demographics, this can be adjusted and optimized with time based on learning on a large population.

In some embodiments system 100 may include a camera such as module 115 that is used to obtain video or still images of the subject to monitor changes in face, physical activity, etc. Camera 115 can be used to obtain pictures of food, beverage, food supplements and/or drugs consumed. Camera module 115 may include image analysis features, while in some embodiments image analysis is performed by processing circuitry 106.

Optionally, system 100 further comprises a learning module (not shown). The learning module may be configured to learn general cognitive scores, physiological parameters and combination thereof from the data obtained from the subject. Alternatively or additionally, the learning module may be configured to learn a correlation of different stored cognitive scores, physiological parameters and combination thereof obtained from a plurality of subjects and use the correlations to identify correlation in the subject. In a non-limiting example, 100 patients performing training X and physiological exercise Y reach a cognitive peak, therefore the individual subject should also perform training X and physiological exercise Y. Optionally, the learning module is a cloud-based learning module.

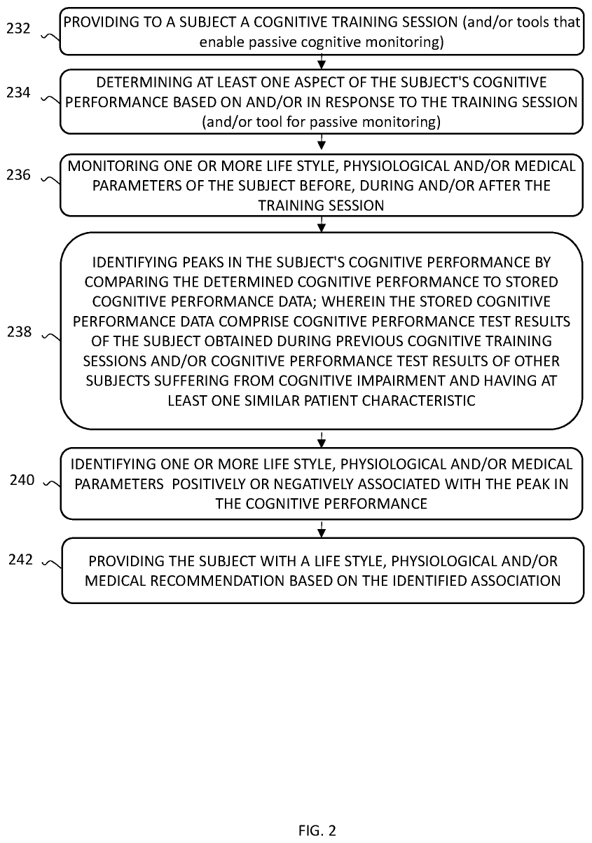

Reference is now made to FIG. 2, which is a schematic flow diagram of a method for managing a subject suffering from cognitive impairment, in accordance with an embodiment. The method may include monitoring cognitive performances of the subject and identifying life style and physiological and/or medical parameters for improving/maintaining the cognitive performance of the subject.

A cognitive training session is provided to a subject (step 232). At least one aspect of the subject's cognitive performance is determined based on and/or in response to the training session (step 234). Optionally, the training is an active training which includes memory training, attention training, lingual training, numeric training, motoric training, social training, reading training, orientation training, problem solving, or any combination thereof. The training characteristics may include: type of training, performances of specific cognitive capabilities and ratio between them, length of training, frequency of training sessions, subject's compliance to the training sessions, or any combination thereof.

One or more life style, physiological and/or medical parameters of the subject are monitored before, during and/or after the training session (step 236). The life style and physiological and/or medical parameters may be measured and/or otherwise obtained. The parameters may be registered, loaded or stored, such as into a data storage unit. The one or more physiologic parameters may be selected from the group consisting of: body temperature, respiratory rate, pulse rate, blood pressure, blood sugar, blood oxygen, cholesterol, blood pH value, body fat, skin resistance, blood pressure, or any combination thereof. The one or more medical parameters are selected from the group consisting of: drug administered, medical treatment, physiotherapy, psychological treatment, psychiatric treatment, or any combination thereof. The one or more life style parameters are selected from the group consisting of: physical activity, nutrition, consumption of food supplements, social interactions, sleep quality, sleep/wakefulness, a degree of maintaining daily routine, or any combination thereof. Further, information such as demographic details, medical history, etc., may be either registered, loaded or stored such as into a data storage unit. Optionally, everyday activities performed by the subject are further monitored.

As many drugs that act on the brain can cause delirium or modulation of cognition, they may be indexed Dᵢ(cⱼ) wherein i is an index from each drug in the list including narcotic painkillers, sedatives (particularly benzodiazepines), stimulants, sleeping pills, zo antidepressants, Parkinson's disease medications, antipsychotics and others drugs such as corticosteroids, cimetidine, digoxin, anticholinergic drugs (including antihistamines and some drugs for digestive problems, allergies, and acute asthma attacks) and muscle relaxants and many potential culprits (some available over the counter). cⱼ is the number of days (or hours when applicable) since the change (use/cessation of use) of drug or dose. For example, a same dose given for more than 30 days may be considered stable dose. Then for each cognitive score change CSCₖ (from base line and/or in recent trend compared to results obtained in the last days) and changes in drugs can be calculated in a kij matrix or representation. For a patient on n drugs and food supplements evaluated for m cognitive scores an array of correlation can be evaluated.

C_{k(1, 2, . . . m)i(1, 2 . . . )}(t,cⱼ)=corr(CSCₖ(t),Dᵢ(cⱼ))

Wherein at a time of evaluation t, the correlation between changes in cognitive score CSCₖ(t) and any drug Dᵢ changed within cⱼ days since t (and others that are on stable dose or changed in other times) is evaluated.

A peak in the subject's cognitive performance is identified by comparing the determined cognitive performance to stored cognitive performance data; wherein the stored cognitive performance data comprise cognitive performance test results of the subject obtained during previous cognitive training sessions and/or cognitive performance test results of other subjects suffering from cognitive impairment and having at least one similar patient characteristic (step 238). Optionally, a score representing the subject's cognitive status is assigned and the score is compared to stored scores. The other subjects may suffer from the same type of cognitive impairment as the trained subject. Other shared patient characteristics may include, age, gender, medical history. Additionally or alternatively, a deterioration in cognitive performance may be further identified, based on the comparison.

One or more life style, physiological and/or medical parameters which are positively or negatively associated with the peak in the cognitive performance are identified (step 240).

A life style, physiological and/or medical recommendation may be provided to the subject based on the identified association (step 242). Examples of recommendations may include, but are not limited to, recommendation to continue a current therapy plan, new medications, increase/decrease dosage of current medications, more/less sleep, more/less exercise, altered dietary components, etc. It is then up to the patient's caregivers or alternatively the patients themselves or may be in combination with family, caregivers, therapists, and so on, to implement the recommendations for a specified period of time.

Any of steps 232, 234, 236, 238, 240, and 242 may be performed in an interchangeable order, in parallel or in sequence.

According to an alternative or additional embodiments, for cognitive training the subject may be provided with tools that enable cognitive monitoring in a passive mode for example, based on voice analysis, analysis of texting, analysis of subject movement, or any combination thereof.

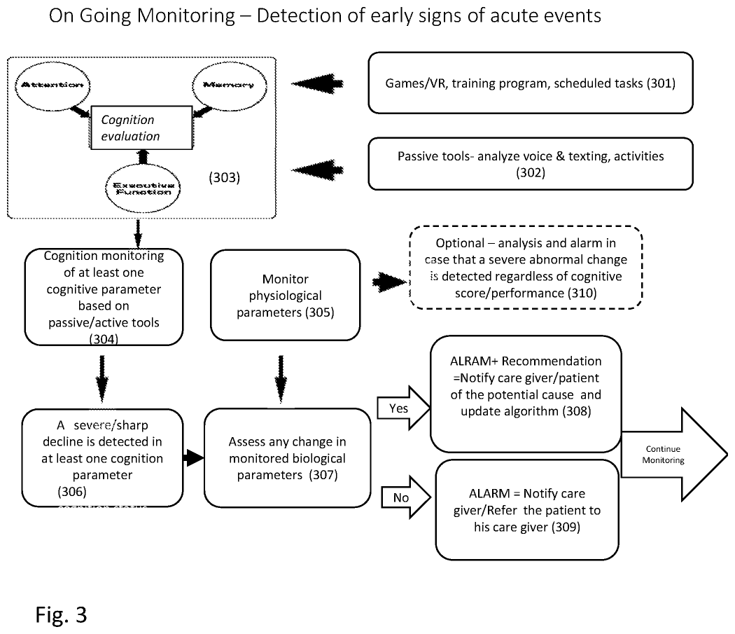

Reference in now made to FIG. 3, which shows a flow chart for (on going) monitoring of patients at risk of developing conditions such as delirium and for detecting signs early enough to prevent such conditions or to improve their outcome, according to some embodiments. In accordance with some embodiments, detection of early signs of acute events is facilitated by the method/system described herein. Various aspects of cognition are evaluated by module 303, based on active tools such as games and responses to active tasks in module 301, or based on passive tools in module 302, such as voice analysis using tools, such as but not limited to, Watson IBM tone analysis. Module 304 monitors the scores obtained from module 303 and feeds module 306 to detect a significant/severe decline in at least one score compared to baseline (such as when patients is admitted to hospitalization or before surgery) and/or to monitor the trend in the scores over time so as to detect gradual/rapid decline. In order to improve sensitivity and specificity of the detection, the analysis can be further based on cloud based data analysis incorporating stored data of the patient or other subject's sharing a similar medical history. Similarly, module 305 monitors physiological related parameters. Module 307 analyzes the potential correlation between signs of cognitive decline and physiological scores/parameters in order to provide/suggest the potential cause that led to an acute event, such as delirium. The module is based on individual data and optionally also based on stored cloud data. The algorithm is, in some embodiments, developed and improved using the data stored in the cloud. When a potential cause/physiological condition/manifestation of abnormal parameters is found, alarm 308 is triggered. In cases that abnormalities in physiological parameters are not detected, or do not appear to correlate with the subject's cognitive score, etc., a more general alarm may be provided (309). In an optional embodiment, module 305 may be connected to an additional module (module 310) configured to detect significant abnormalities and to trigger alarm in case of sever abnormal findings. In some embodiments, the earlier/dominant trigger of the alarm is based on physiological changes whereas cognitive changes may be secondary (i.e., complimentary to the scheme present in FIG. 3)

Optionally the systems of the present disclosure may include modules that can evaluate the subject and select and/or tailor the training and/or cognitive monitoring tool according to the subject's capabilities and status and can be modified according to his/her status. According to some embodiments, a similar selection of the physiological parameters to be collected and means to do that (such as the appropriate sensors) is selected and tailored according to the subject condition. For example, a different set of data may be appropriate when a patient is admitted to hospital or surgery (and includes daily blood tests) vs. data that needs to be collected in a community setting (that may include for example glucose sensing), wherein other medical conditions are more likely to lead to cognitive deterioration.

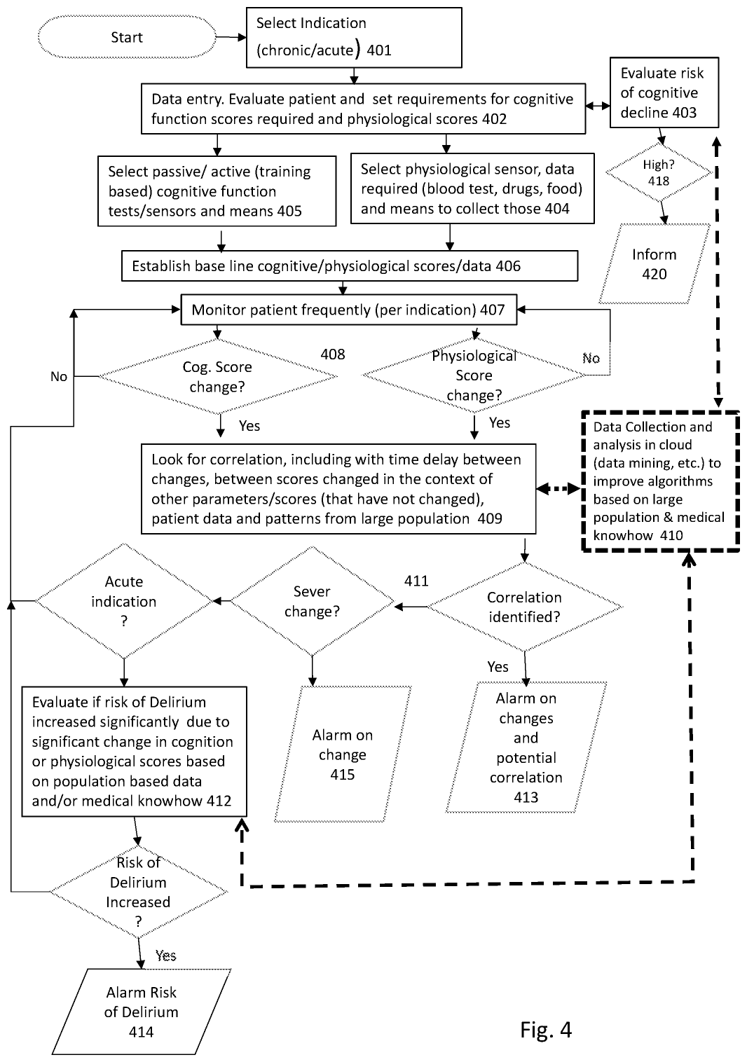

Reference in now made to FIG. 4, which illustrates a high-level flow chart of a system that aims to avoid or minimize/avoid cognitive drops. This flow chart covers both indications: delirium (acute cognitive impairment) prevention and, in chronic cognitive impairment, avoiding/minimizing cognitive drops and enhancing cognitive peaks for the benefit of subjects/patients, for example, elderly/patients with neurological disorders.

According to some embodiments, in delirium four (4) stages may be defined for the purpose of this disclosure. The systems and methods provided herein, according to some embodiments, aim to prevent patients from developing delirium and avoid a significant acute cognitive drop and/or improve prognosis by early detection of drop and/or zo physiological abnormality that triggers it:

Phase 1—Patient is hospitalized/admitted to surgery. The systems and methods disclosed herein, according to some embodiments, advantageously facilitate assessment of risk of delirium and provide means to avoid it by, for example, providing instructions on how to deal with the patient, etc.

Phase 2—Patient is about to develop delirium but even an expert would not diagnose him/her as suffering from delirium. Patients are likely to suffer from delirium in a matter of days/hours. The systems and methods disclosed herein, according to some embodiments, advantageously facilitate identifying the cause of the delirium and managing it, thus preventing/ameliorating the delirium.

Phase 3—Patient develops delirium but clinical manifestation is not clear/strong. While, usually, patients with hyperactive delirium demonstrate features of restlessness, agitation and hyper vigilance, and patients with hypoactive delirium seeming to be in a daze and show little spontaneous movement, the clinical appearance may not be clear so caregivers who are not experts may miss it. The systems and methods disclosed herein, according to some embodiments, advantageously facilitate identifying that a patient is developing delirium early enough so that the patient can be treated and his/her prognosis may be improved.

Phase 4—Clear signs of delirium. The systems and methods disclosed herein, according to some embodiments, may further validate the diagnosis.

For chronic indication, even in cases where prevention of every single cognitive drop is not achieved, the systems and methods provided herein, in accordance with some embodiments, still allow identifying causes/triggers for drops and/or elevated function to provide recommendations to improve cognitive function in the relevant population.

Accordingly, the method/use of the system, as disclosed in FIG. 4, starts with identification of the indication, 401 (for example, chronic vs. acute condition). Data of the patient/subject demographic, physical and cognitive status, etc. is loaded and evaluated and according to the characteristics of the specific subject, the system recommends a list of parameters that need to be monitored to evaluate cognitive/physiological scores/changes, 402. The system performs an initial evaluation of risk of cognitive drop, 403, such as high risk to develop delirium in specific elderly patients admitted for hip replacement, phase 1, above. The system can provide such analysis, quantitative prognosis based on information/clusters developed from a large population analysis and/or medical know how analysis, 410. 410 may be performed in external servers while 402 (and similarly, 405, 404, 409 etc.) may be performed by a local processor or also by cloud based analysis.

In case a significant/high risk is identified, 418, a caregiver/individual/subject is/are informed 420.

Based on the analysis of 402, sensors are selected to monitor and enable cognitive scores 405 to be provided. For example, for patients with a high cognitive baseline, a different balance between active (training based) vs. passive may be selected. Also, for patients that are at risk to develop delirium, scores of alertness are more relevant than memory, in which case standard training of memory is less relevant. Similarly, the physiological scores, 404, for example, for prevention of delirium, EEG monitoring may be selected for daily monitoring while less relevant for chronic use. Blood tests may be also relevant for delirium and less relevant in chronic settings. On the other hand, a sensor that monitors movement may be relevant for both indications. The same applies for food and liquid consumption, and some standard parameters such as, but not limited to, body temperature, blood pressure and SpO₂. Sensors known in the art may be used to interface with the system.

The selected sensors start to work with a collection of baseline data, 406. This can be stored for reference to follow changes in the individual of interest. Depending on the indication, continuous/continual/periodic monitoring starts, 407. In delirium, indication frequency should typically be higher, preferably at least three times per day due to the fast course of this condition, while in a chronic setting indication frequency can be lower. The system looks for significant cognitive changes, 408. For data that is not quantitative/represented in numbers by nature such as food, “mathematical” representation may be established to monitor changes. When significant change/changes in cognitive and/or physiological scores is/are identified by 408, the system, 409, (analysis can be performed locally and/or in the cloud) looks for correlation/association between changes. As disclosed above, a time delay between changes may be expected and evaluated. In chronic/routine use indication, correlation evaluation may be assisted by historical data that helps to identify physiological causes that lead to fluctuation in cognition as episodes of sleep problems, certain foods/food supplements that elevate/impair cognition and drugs. To improve evaluation, analysis may be assisted by patterns/algorithm developed/identified based on big data analysis and medical know-how, 410. Accordingly, performances are expected to improve by data provided from individual analysis 409 to the analysis and know-how in the cloud 410.

Based on the analysis, several questions are asked 411: If correlation is identified, the system provides an output about the changes and potential correlation (and optionally an alarm), 413. Depending of the output, the information may be provided to the individual and/or as data for the caregiver. When correlation/association is not clear but still the change is significant, an alarm is provided 415. Even if change is not severe but the aim is to avoid delirium, the risk for development of delirium is evaluated by module 412. For example, if the system identifies signs of infection such as in the urinary tract that are quite common in hospitalized patients, it will generate an alarm that a risk of delirium increases, 414, also if no significant change in cognitive function as been identified. If, on the other hand, there is a drop in alertness or a drastic change in movement of the patient, the system will trigger an alarm even when no other change was identified. To perform this analysis and provide alarms at early stages (for example, such as described in phases 1-3 above) with high sensitivity/specificity (or other accuracy measures) big data analysis is performed using data science methodologies 410. The analysis can include retrospective analysis of data collected from patients who developed or have not developed delirium during hospitalization.

The alarms and information about potential correlation between physiological changes and cognitive changes can be also provided through clinical decision support systems or HER/EMR systems. Those tools are known in the art https://en.wikipedia.org/wiki/Clinical_decision_support_system

In the description and claims of the application, each of the words “comprise” “include” and “have”, and forms thereof, are not necessarily limited to members in a list with which the words may be associated.

The present invention may be a system, a method, and/or a computer program product. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, or either source code or object code written in any combination of one or more programming languages, including an object oriented programming language such as Java, Smalltalk, C++ or the like, and conventional procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server.

In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the block may occur out of the order noted in the figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

While a number of exemplary aspects and embodiments have been discussed above, those of skill in the art will recognize certain modifications, permutations, additions and sub-combinations thereof. It is therefore intended that the following appended claims and claims hereafter introduced be interpreted to include all such modifications, permutations, additions and sub-combinations as are within their true spirit and scope.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

1. A computer implemented method for managing a subject suffering from cognitive impairment, the method comprising:
providing to a subject a cognitive training session;
determining at least one aspect of the subject's cognitive performance based on and/or in response to said training session;
monitoring one or more life style, physiological and/or medical parameters of the subject before, during and/or after said training session;
identifying peaks in the subject's cognitive performance; wherein the identifying comprises:
comparing the determined cognitive performance to stored cognitive performance data; wherein the stored cognitive performance data comprise cognitive performance test results of the subject, obtained during previous cognitive training sessions and/or cognitive performance test results of other subjects suffering from cognitive impairment and having at least one similar patient characteristic;
identifying one or more life style, physiological and/or medical parameters positively or negatively associated with the peak in the cognitive performance; and
providing the subject with a life style, physiological and/or medical recommendation based on the identified association.

2. The method of claim 1, wherein the other subjects are suffering from the same type of cognitive impairment as the trained subject.

3. The method of claim 1, wherein the cognitive impairment is associated with Alzheimer's disease.

4. The method of claim 1, further comprising:
providing to the subject a second training session, and determining the at least one aspect of the subject's cognitive performance based on and/or in response to said second training session; and
comparing the cognitive response obtained in response to said first and second training sessions and determining one or more training characteristics associated with a better cognitive performance,
wherein the one or more training characteristics comprise type of training, performances of specific cognitive capabilities and ratio between them, length of training, frequency of training sessions, subject's compliance to the training sessions, or any combination thereof.

5.-6. (canceled)

7. The method of claim 4 wherein the first and second training sessions are different from one another.

8. The method of claim 1, wherein the one or more physiologic parameters are selected from the group consisting of: body temperature, respiratory rate, pulse rate, blood pressure, blood sugar, blood oxygen, cholesterol, blood pH value, body fat, skin resistance, blood pressure, or any combination thereof,

9. The method of claim 1, wherein the one or more medical parameters are selected from the group consisting of: drug administered, medical treatment, physiotherapy, psychological treatment, psychiatric treatment or any combination thereof.

10. The method of claim 1, wherein the one or more life style parameters are selected from the group consisting of: physical activity, nutrition, consumption of food supplements, social interactions, sleep quality, sleep/wakefulness, degree of maintaining daily routine, or any combination thereof.

11. The method of claim 1, further comprising assigning a score representing the subject's cognitive status.

12. The method of claim 1, further comprising identifying, based on said comparison, a deterioration in cognitive performance and providing the subject with a life style, physiological and/or medical recommendation based on the identification.

13. The method of claim 1, wherein the training is an active training comprising memory training, attention training, lingual training, numeric training, motoric training, social training, reading training, orientation training, problem solving, or any combination thereof.

14. The method of claim 1, further comprising monitoring every day activities performed by the subject.

15. The method of claim 1, further comprising identifying every day activities positively or negatively associated with the peak in the cognitive performance; and providing the subject with a recommendation based on the identified association.

16. The method of claim 1, further comprising recording and/or storing the subject's memories during periods of peak performance.

17. (canceled)

18. A system for managing a cognitive condition of a subject suffering from cognitive impairment, the system comprising:
a cognitive monitoring unit configured to monitor the subject and to determine a change in the subject's cognitive performance;
one or more sensors configured to monitor one or more life style, physiologic and/or medical parameters of the subject before, during and/or after said monitoring period; and
a processing circuitry configured to:
identify peaks in the subject's cognitive performance; wherein the identifying comprises comparing the determined cognitive performance to stored cognitive performance data; wherein the stored cognitive performance data comprise cognitive performance test results of the subject obtained during previous cognitive monitoring periods and/or cognitive performance test results of other subjects suffering from cognitive impairment and having at least one similar patient characteristic,
identify one or more changes in life style, physiological and/or medical parameters positively or negatively associated with the peak in the cognitive performance; and
provide the subject with a life style, physiological and/or medical recommendation based on the identified association.

19. The system of claim 18, wherein the other subjects are suffering from the same cognitive impairment as the monitored subject.

20. The system of claim 18, wherein the cognitive impairment is associated with Alzheimer's disease.

21.-36. (canceled)

37. A computer implemented method to avoid or minimize cognitive drops, the method comprising:
providing to a subject a cognitive monitoring tool, the cognitive monitoring tool comprising at least one passive monitoring and/or training session;
determining at least one aspect of the subject's cognitive performance based at least on the cognitive monitoring tool;
monitoring one or more life style, physiological and/or medical parameters of the subject before, during and/or after the passive monitoring and/or training session;
identifying a decline in the subject's cognitive performance; wherein the identifying comprises:
comparing the determined cognitive performance to stored cognitive performance data; wherein the stored cognitive performance data comprise cognitive performance test results of the subject obtained during previous cognitive sessions;
identifying changes in one or more physiological and/or medical parameters positively or negatively associated with the decline in the cognitive performance; and
providing an output signal indicative of one or more physiological and/or medical parameters positively or negatively associated with the decline in the cognitive performance.

38. The method of claim 37, further comprises providing an alarm.

39. The method of claim 37, further comprises providing a physiological and/or medical recommendation based on the identified association.
